(19) United States
(12) Patent Application Publication
(10) Pub. No.: US 2003/0072750 A1
(43) Pub. Date: Apr. 17, 2003

(54) Compositions and means for the treatment of burns and other cutaneous traumas

(75) Inventor: Lior Rosenberg, Omer (IL)

Correspondence Address: ANDERSON KILL & OLICK, P.C., 1251 Avenue of the Americas, New York, NY, 10020, US

(73) Assignee: L.R. R & D, Ltd., Omer (IL)

(21) Appl. No.: 10/293,461

(22) Filed: Nov. 12, 2002

Related U.S. Application Data
(62) Division of application No. 09/449,094, filed on Nov. 24, 1999, now abandoned.

(30) Foreign Application Priority Data
May 26, 1997 (IL) 120909

(51) Int. Cl. A61K 38/48; A61K 9/70

(52) U.S. Cl. 424/94.63; 424/443

(57) ABSTRACT

Disclosure relates to a unit dose debriding matrix carrier, comprising a housing containing lyophilized or otherwise dried debriding agent, the housing being made of porous material so as to allow passage of the debriding agent therethrough when a liquid is applied thereto, the carrier further having a polygonal shape such that a plurality of identical carriers can be placed around it, to fill a wound area. This application also relates to a placing device for a unit dose debriding matrix carrier, comprising elastic holding means to hold the matrix in place within the device and to release the carrier when a light pressure is applied on the device, and a unit dose powder/vehicle-carrier-gel mixing and placing device comprising unit dose powder and vehicle containers and means for joining said containers and mixing the countenance aspeticly and extracting the mixture onto the wound.

### FIELD OF THE INVENTION

The present invention relates to the treatment of traumatized skin. More particularly, the invention relates to compositions and means for promoting the healing of skin by a suitable selective eschar removal and wound healing promotion.

### BACKGROUND OF THE INVENTION

Trauma to the skin is the commonest trauma in humans. The trauma may be acute or chronic due to any kind of offensive physical agent (such as thermal, chemical, pressure, shearing, degloving etc.). Due to its sudden onset, severity, complexity, short and long term implications the cutaneous burn trauma will be used here as an example to other cutaneous traumas.

The burn is one of the most severe and dreaded traumas in the modern, developed parts of the world, and even more so in its less developed areas. Up to 100,000 American are severely burned each year and need specialized, intensive burn unit facilities for their treatment. More than a million Americans each year are treated in general surgery or other non-specialized medical units. The numbers of small burns are practically unfathomed and may be estimated to be far beyond 10,000,000 each year.

The traumatized skin (burned tissue) the eschar, may be of different depths, including parts or the entire thickness of the skin and even other, deeper tissues. The eschar characteristics may depend on the traumatizing agent (thermal, chemical and electrical), the eschar's age and the conditions that influenced the eschar since the onset of the trauma. Leaving the dead eschar in place will extend and deepen the damage into the neighboring, originally undamaged tissues. This dead eschar will serve as a medium for bacteria growth, and a source of infection, contamination and sepsis that may lead even to the patient's death. Some modern studies highlight the relation between the presence of the eschar and deterioration of the general immune and resistance systems, a phenomenon that promotes the oncoming sepsis.

The assessment of the primary tissue damage in burns is difficult. The burn's depth is changing from point to point and the eschar' color and texture may be misleading even to the expert's eye. Very often the burn depth may be determined only after few days, when the secondary damage already extends beyond the original burn eschar. In order to prevent the above mentioned complications, it is imperative to remove at the earliest stage, as much as possible of the offending eschar. This removing of the dead tissue is termed “debridement”. The concept of debridement is as old as medicine itself but its execution is extremely difficult and not free of risks.

The most obvious and direct debridement method is surgery. In small, limited necrotic areas excision of the entire dead tissue up to healthy, bleeding tissues is the procedure of choice. In the case of burns, because of the large surfaces of dead eschar, a tangential excision (with the help of special knives called dermatomes) of the dead eschar, layer after layer is done. The excision should be carried down into the healthy intact tissue to make sure that no trace of the dreaded eschar remains. It is estimated that up to 30%-50% of healthy tissue may be sacrificed in this procedure. This healthy tissue, if preserved, could serve as a source for the natural healing processes. These surgical procedures are long, difficult, demanding of patient's and medical resources with profuse bleeding. Surgery was and still is the most common debridement technique but because of its cost and implications should not be chosen lightly without a critical assessment of each patient and each burn site.

The raw surface that is left after the thorough debridement should be protected and covered immediately to prevent desiccation and further tissue death. Due to the depth of the surgical debridement, grafting with autogenous or non-autogenous grafts is practically the only answer. The harvesting of the skin graft demands extending surgery into other healthy skin areas intact until now. In large burns, only few and precious potential donor sites exist. Thus, hard to find omografts or expensive (and not too effective) synthetic skin substitutes or biological dressings are used.

Historically, the burn area was defined appropriately debrided only if it could host a living skin graft. This is achieved by removal of tissue deeper than the eschar, which results in heavy bleeding of the wounded healthy tissue. The bleeding itself serves as the sign of appropriately debrided wound, assuming that tissue that does not bleed heavily will not be able to support a skin graft. The severed blood vessels are the ones that will grow into the graft and provide the necessary blood supply necessary for its survival. The appearance of the bleeding bed and its “graftability” is crucial in defining the debridement efficacy according to the known art. In fact, one of the problems encountered with the chemical debridement of wounds is that some debriding agents have been known to be unsuitable for therapy, because they did not leave a heavily bleeding tissue bed, and therefore caused the failure of skin grafting. The appearance of the surgically debrided tissue is typical and made of healthy dermal collagen, subdermal tissue and vessels all transected by the tangential excision

Due to the abovementioned difficulties (of treating large traumatized body areas of a very unstable patient, difficult diagnosis of the damage's extent, extensive and risky surgery with immense blood loss, need for huge quantities of grafts (that is only rarely available) there have been always a tendency to continue a “conservative” treatment of the burned areas leaving the burn eschar in place. If the eschar is left untreated, autolisis and decomposition due mainly to the activities of the growing germs within the dead eschar will lead to what was coined as “spontaneous sloughing” (not unlike the “laudable pus” of old). Obviously this phenomenon is nothing more than an immense purulent, inflammatory reaction on large areas of the immune compromised patient's body. The violent sepsis and inflammation process leads to propagation of tissue damage inward: transforming second degree and partial thickness damage into a full thickness third degree one. The sloughing phenomenon takes 2-4 weeks while the patient is severely, and some times terminally, septic. If the patient survives the ordeal he is left with most of the originally burned areas completely exposed and raw or covered with granulation tissue that eventually will evolve (even if grafted at this stage) into deforming and contracted scar tissue. In order to treat the dangerous inflammatory stage several topical treatment modalities, using topical antiseptic or antibiotic preparations were developed (some with rather severe side effects). Some of these treatments do partially prevent some of the inflammatory and sepsis problem but with a price: A severe delay of the eschar sloughing with increase granulation/scar tissue formation and late scarring sequels and increase in healing time.

The idea of using a debriding enzymes or chemical agents to “dissolve” the burn eschar and to prevent at least the severely traumatic surgical debridement is not new. Ideally, it has been postulated that one would wish for an ointment or other local preparation that could be easily applied, as soon as possible on the fresh burn without extending the original trauma and harming undamaged tissues. This ideal debriding agent should separate quickly and selectively only the damaged tissues leaving an intact raw surface that could support a skin graft.

Many chemicals with proteolitic activity such as Salicilic acid, Benzoic acid, Malic acid (Aserbin), Collagenase (Varidase, Santyl), Trypsin (Trypur) and Fibrinolysin-desoxyribonuclease (Elase) compounds were or still are in current clinical use since the second world war. Several enzymes, of microbacterial, vegetable or even animal origin were tested and some even reached the market. These enzymes derives from microorganisms such as Bacillus subtilis: Sutilains (Travase), Streptococci: Streptokinase-streptodornase, plants such as the Papaya (Papain) or Bromelain from the Pineapple (Debridase, Escharase, Ananain). Even enzymes made of krill or pancreatic powder were tried. Most of these compounds demand one or two daily dressing changes for five to ten days. By the time the entire eschar is removed a rich granulation tissue has been developed in many areas with a future scar and contracture formation. Unfortunately all of them, including the newly developed ones such as Travase and Genzyme Ananain require several daily dressing changes for no less than five and sometimes more than twelve days. The use of these enzymes was followed in several cases by a violent sepsis and even septic shock probably due to bacteremia from exposure of the raw tissues to several days old, contaminated and partially dissolved eschar. The debrided tissue could not support an autogenous or non autogenous living graft without an additional surgical debridement, thus, the debrided tissue underwent a secondary damage through desiccation and exposure with a secondary increase of the tissue necrosis.

Based on the above-mentioned data that represent the “state of the art” of burn treatment the following choices of burn treatment protocols may be considered to date:

1. The ages-old “conservative” supportive treatment of the eschar using locally applied antiseptic and antibacterial preparations that results in a late spontaneous eschar sloughing (two-four weeks), accompanied often by sepsis and resulting in granulation tissues, the originator of heavy and deforming scars. Once the burn is clean of the eschar and a healthy granulation tissue has been developed, an autogenous skin should be grafted to prevent the late sequel. The area that needs to be grafted is usually most of the entire second and third degree burn (the second and mixed depth burns has been transformed into deep burns through the secondary burn propagation and infection/inflammation process).

2. The “surgical” approach that consists of a tangential excision of the eschar that sacrifices healthy tissues and needs immediate biological coverage and grafting to prevent transformation of the new clean raw debrided tissue into a new necrotic one. This protocol shortens hospitalizationlhealing time and when performed early enough may prevent sepsis and scar contraction. The drawbacks are that the surgery is extensive, risky, with a very heavy load on the hospital resources, facilities and highly trained manpower. The need of an immediate grafting of the raw debrided tissue dictates usually an autograft with an additional increase of at least 10-15% of the exposed, denuded TBSA (Total Body Surface Area) or a scarce and costly omograft or substitutes. Due to the present surgical techniques the debrided area that need grafting is usually most of the second and all the third degree burns.

Thus, in both treatment modalities the final tissues death and necrosis and skin defect that follows is more extensive than the original traumatized tissue.

### SUMMARY OF THE INVENTION

It has now been found, And this is an object of the present invention, that it is possible to provide means for treating wounds of the type described above, without suffering from the disadvantages of the state of the art treatments. The new treatment means are based on the discovery that both the above mentioned traditional treatment modalities are based on two opposing misconceptions. The first is an undertreatment, leaving the dead eschar on the patient with all the severe, often lethal, drawbacks, complications and sequels. The second is an overtreatment, debriding aggressively the dead eschar but paying dearly with blood, precious healthy tissues, dangerous procedures and the necessity of grafting immediately the exposed, raw, debrided areas.

It is an object of the invention to provide biologically active compositions for the debridement of dead eschar, which leave what will be termed hereinafter an “interface layer” (I.L.) between the dead, necrotic eschar and the entirely normal unharmed (graftable) tissues. This interface layer, achieved by a suitable enzymatic/chemical debridement, is characterized by a rather normal looking collagen fibers and anatomical microstructure but with very few open blood vessels (such as encountered in a surgically debrided wound where the level of incision is entirely within the normal tissue). The behavior of this interface layer is different compared to the dead eschar or to the surgically debrided wound. The absence of dead, necrotic tissue prevents secondary germ's contamination and sepsis. Its structure does not tend to readily receive a skin graft and when applied, it may survive only for very few days due to the first stage of “graft tack” that is a passive serum imbibition. The second and definite stage of neo-vascularization does not proceed as readily in the interface layer as in the surgically debrided wounds because of the relative poverty of open blood vessels. In surgically debrided wounds the open vessels eventually support the graft with their host/graft direct anasthomosis or budding potential.

Otherwise, this interface layer, if protected from desiccation or heavy contamination and treated in the correct way, exhibits a remarkable potential for a spontaneous reepithelialization and healing. Once the epithelial remnants in the skin adnexae are given the right conditions for proliferation and propagation, the newly debrided collagen bed provides adequate conditions for a fast reepithelialization. Fast (less than three weeks) epithelialization prevents the formation of the granulation tissue that eventually develops into heavy and contracted scar tissue.

Once all the dermal remnants are epithelializated (in the cases of second or mixed depth burns) only a small percent of the originally damaged skin (usually part of the full thickness, deep-or third degree burns) remains to treat. Correctly treated, by the end of second to fourth week post-burn, most of the burn is healed by epithelialization and the few, relatively small areas that are not epithelializated are clean, free of necrotic tissue and have an adequate capillary bed to support and host a graft. At this stage, with the patient's general condition improved, these areas may be grafted by autogenous graft. Obviously, as the grafted area is rather small, the donor site areas and the extent of the graft harvesting and grafting procedure is very limited.

The conditions for the preservation of the newly debrided skin and provision of the condition of fast, spontaneous epithelialization depends on the right cover (such as the natural split thickness graft) of the row, debrided areas.

The required features of this cover are as follows:

Readily available after debridement

Adheres to the exposed, debrided areas

Allows epithelial propagation along and under its structure

Provides the right physical conditions (temperature, humidity, etc.) for the wound healing process

Optionally provides also the important growth factors for wound healing and epithelialization process.

Thus, the main advantages of the treatment made possible by the invention are as follows:

1. Early, complete debridement of all necrotic tissues.

2. Non-traumatic, bloodless and low-risk non-surgical debridement.

3. Accurate early assessment of damage extent (depth and surface).

4. Natural fast epithelialization of most of the burn surface with little or no scarring.

5. Grafting of only small part of the original burned area.

6. Fast epithelialization and grafting of the debrided areas prevents scar formation.

7. Very cost effective compared to the state of the art treatment modalities.

The clinical implications of these advantages are:

1. prevention of tissue secondary damage propagation.

2. preservation of all viable tissue components.

3. prevention of sepsis due to tissue necrosis.

4. early diagnosis of the extent of tissue damage.

5. early enhancement of “spontaneous” skin healing wherever it is possible by a maximum exploitation of the tissue's entire regenerative potential.

6. autogenous grafting exclusively of the remaining full thickness defects.

Thus, the present invention provides, inter alia, a skin pre-healing composition, for the pre-treatment of traumatized skin, comprising an interface layer-forming effective amount and application means of a debriding agent. The debriding agent is present in an amount and nature that does not interfere with unharmed tissue under or around the eschar, or induce substantial bleeding after debridement is completed. The debridement does not harm or dissect the normal dermis or its collagen/elastin fibers.

According to one preferred embodiment of the invention, the debriding agent comprises one or more enzymes. In another preferred embodiment of the invention the debriding agent is derived from pineapple. Typical debriding agents of this kind include, e.g., Bromelain or a derivative or fraction thereof, such as Debridase, Escharase or Ananain.

In another aspect, the invention is directed to an early coverage set for the protection of an interface layer of a wound debrided by a composition as described above and promotion of its healing, comprising a protective dressing that may be provided with Keratocyte growth promoting agent(s).

According to a preferred embodiment of the invention, the Keratocyte growth promoting agent comprises an artificial dermis. According to another preferred embodiment of the invention, the Keratocyte growth promoting agent comprises one or more growth hormones.

The invention further provides a method for treating a patient suffering from trauma of the skin, said method comprising the steps of:

(a) pre-treating the wound by humidification;

(b) treating the wound with a debriding agent in an amount and for a period of time that leave the untraumatized tissues unharmed, do not promote substantial bleeding and/or contamination, and which generate an interface layer, as defined herein;

(c) covering the debrided wound with a matrix and layer which promotes keratocytes propagation, for a period of time sufficient to permit spontaneous healing of the interface layer; and

(d) grafting areas of deeper wound which were not healed through keratocytes propagation as described in (c) above.

Preferably, but non-limitatively, the debridement procedure is carried out for a period of time that does not exceed 4 hours. Keratocyte propagation, in turn, is allowed to proceed for about 2 to 4 weeks.

Other objects and advantages of the invention will become apparent as the description proceeds.

### BRIEF DESCRIPTION OF THE DRAWINGS

The above and other characteristics and advantages of the invention will be more readily apparent through the following detailed description of preferred embodiments thereof, with reference to the appended drawings, wherein:

FIG. 1 is a photography of a fresh, mixed depth, scaled burn of the right thigh, 2% circa TBSA. Most of the keratin blister has been removed, the pink-reddish periphery numbered 1 (seen in greyscale in the figure) seems to be of a second-superficial depth. The center, white-gray area 2 is deeper and is of a second-deep (deep-dermal) depth.

FIG. 2 is a photography of the same burn of FIG. 1 after an enzymatic debridement (Debridase for 4 hours). The periphery of the more superficial mid depth burn shows some small capillaries punctuate bleeding, area 3. The central deeper area 4 shows the typical aspect of the I.L. of a second-superficial (mid depth) burn with an abundant dermis preserved. Few punctuate bleeding vessels with no active, intense bleeding can be seen. One may note the typical granular aspect of the I.L. due to the irregularity of the original damage: the tissue around the skin adnexae is better preserved and the epidermal remnants there presented are the source for the future healing and epithelialization process.

FIG. 3 is a photography of a deep (nominal third degree) burn of the arm and forearm. The burn was enzymatically debrided (Debridase for 4 hours). At the lateral and posterior aspect there are two islands of non debrided tissue that shows clearly the eschar thickness and its typical yellow-gray color where the original keratin exists (numbered 5) and white area 6 where the keratin was peeled off and only the full thickness dermal eschar is present. The whitish areas marked 7 shows clearly a very thin I.L. with its typical granular pinkish aspect and slightly bleeding capillaries. At the center, area marked 8 is a full thickness burn with the viable fat and thrombosed vein that shows under the I.L. 7. At the right hand side, the elbow area numbered 9 is of a somewhat thicker I.L. and a less damaged dermis.

FIG. 4 is a photography of a wider field and general appearance of the FIG. 3 burn. One may see areas 5 and 6 of the non debrided eschar, areas 7 of a deeper burn and thin preserved I.L., area 9 of a thicker I.L. (second deep or deep dermal burns) and areas 10 of a more superficial burns, a thicker I.L. with the typical punctuate capillary bleeding similar to area 3 in FIG. 2 due to the preserved dermal papillary layer.

FIG. 5 is a photography of a deep mixed flame burn (similar in nature to the burns in FIGS. 3 & 4) after a formal tangential excision. It is evident that the nature of the debrided dermis (area 11 where the blood was wiped dry) is different from the I.L. It is smooth and shiny in comparison to the rather opaque and granular nature of the I.L. The profuse bleeding of the surgically excised skin is apparent in comparison to the sluggish, punctuate, capillary bleeding that stops spontaneously after a few seconds.

FIG. 6 is a drawing of a soaking dressing whereas 12 represents the dressing that is of an occlusive type (such as the M.D.O.D.) or an open, absorbent material that moisturizes the wound's surface by capillary transfer. Container 13 represents the germ-free soaking liquids that may be within infusion bag or other form of container connected to the dressing by tube 14. If a vigorous soaking is required, a draining tube 15 drains the access fluids from the wounds site into a collecting container 16.

FIG. 7 is a schematic drawing representing the piglet bioassay site (see below) whereas 17 represents the healthy, nontraumatized skin, 18 is the area of a mixed depth burn where it is more superficial at the periphery and deeper as it approaches the center. The central area numbered 19 is the deep, full thickness burn. The lenticular biopsy excision 20 represents all the different areas (17, 18 and 19) of the assay.

FIG. 8 is a drawing representing the biopsy (FIG. 7 no. 20) where the different zones are represented in their cross section. Zone 21 is the healthy skin, 22 is the rather superficial mixed (second degree) depth burn, 23 is the deeper second degree (deep dermal) burn and 24 is the full thickness, third degree burn.

FIG. 9 is a drawing of a unit dose debriding matrix carrier saturated with lyophilized enzyme, with an optional rigid frame 125 made of inert materials such as plastic as in FIGS. 10 and 11.

FIG. 10 illustrates a placing device for the matrix carrier, with or without the frame 225, in cross section. When pressure is applied to the handles 41, one towards the other, possibly with one hand, the catches 42, are moved apart from each other and the unit dose debriding matrix carrier, 40, is released.

FIG. 11 is a drawing of a placing device for the matrix carrier in cross section. Said device comprises a cartridge, 43, which contains one or more unit dose debriding matrix carriers, 44, may be separated one from the other by optional protecting disks, 25. These disks may be in the form of a rigid frame with large openings for the passage of the debriding agent's solvent or may be a part of the matrix carrier itself designed to contain the debriding agent and provide rigidity when needed. Pressure is applied on the matrix carriers and protecting disks towards the opening of the cartridge, by a spring, 26. A spring, 29, applies pressure on a catch, 28, and thus said catch is held in place to prevent the release of the disks and matrix carriers. The spring loaded trigger, 27, is pulled and the catch is moved in such a way that either one disk or one matrix carrier is released. Thus, a matrix carrier can be released from a protected, sterile container and placed into an accurate position using one hand.

FIG. 12 is a drawing of a unit dose, uniform, dispersal device in cross section. Said device comprises a cartridge, 30, which contains Debridase or other debriding powder, 31, and a spring, 32, which applies pressure on a disk, 33, which transfers pressure to the debriding powder towards the opening of the cartridge. The bottom side of the cartridge is a flat plate, 34, movable in linear reciprocal motion, in the directions of arrow 35. By pulling the spring loaded trigger, 27, said side is moved in a full cycle, i.e. in such a way that the “peeling blade”, 36, is moved from close to side 37 to close to side 38, and back to close to side 37. At the first half of each cycle, i.e. when said “peeling blade” is moved from close to side 37 to close to side 38, a uniform layer of powder, consisting a constant quantity of powder, is “peeled” and released to the outside of the cartridge, and covers a rectangular area beneath said cartridge with a homogeneous layer of powder. The “peeling blade” 36 may be in the form of a “peeling cylinder” that by turning releases a predetermined quantity of powder.

FIGS. 13a and 13b are drawings of an example of a disposable, unit dose, mixing device-system. FIG. 13c is a schematic partial cross-section of the round inlet 50 as herinafter described. Said system comprises a tubular container 39 containing dry debriding powder. Said container has an enlarged lower end 140 closed by a peel off film 141. A special plunger 142 is placed within the tubular container and has an inferior extension rod 143 with two pairs of flexible, hydrodynamic propelling stirring arms 144 (the superior) and 45 (the inferior one). On the superior part of the plunger a handle 46 join the plunger by a bi-directional joint 47 and to the other end of the handle a “T” like jointed pressing cross bar 48. Another component of the system is a round container 49 for the debriding powder, aqueous or liquid vehicle, solvent or activating medium gel. On its top there is a round inlet port 50 that fits inside the lower extended end 140 of the tubular container 39 and covered with a peel off film 51, and an inner ledge or a groove 52 in the inner surface of said port that engages the plunger 142. A second outlet port in the center of the inferior container surface is closed with flap or cover 54. Two elastic or other kind of clips 55 & 56 on the container's 49 external wall hold the tubular container of the debriding powder in place.

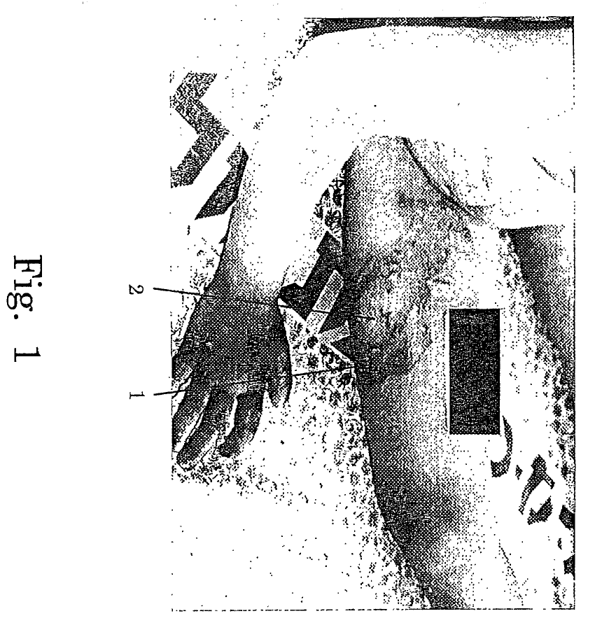

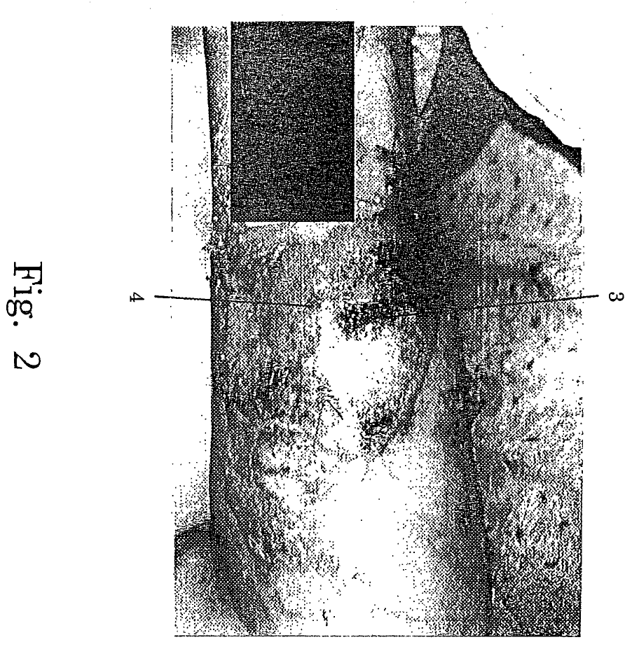

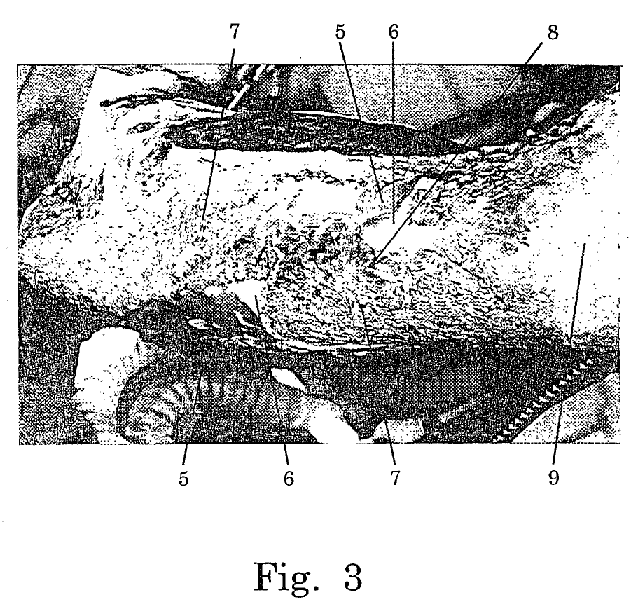

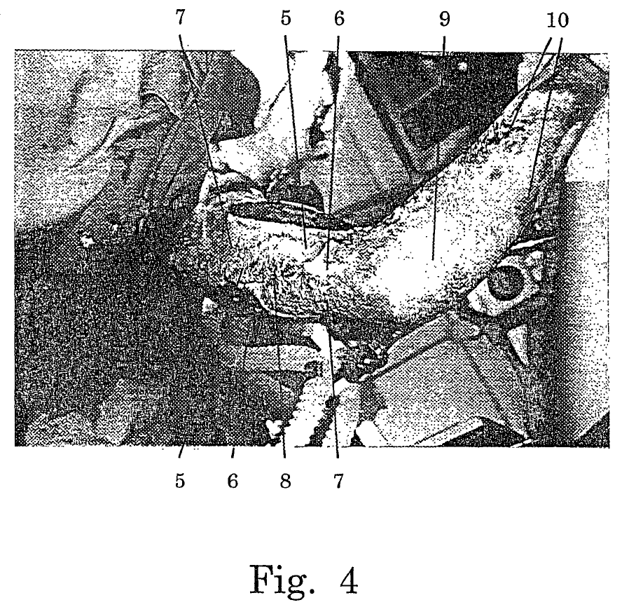

The quantity of the debriding agent and the solvent vehicle may be precisely predetermine. After unclipping the tubular container 39 from the holding clips, the peel off films 141 and 51 are removed and the extended end 140 engages the superior, inlet port 50. After extending-straightening the handle 46 it is pressed downward by pressing the pressing surface 48 of the cross bar and the plunger being pressed down pushes-extracts the debriding powder from its container into the solvent gel. The plunger reaches and locks into the groove 52 and the two pairs of the stirring arms 144 & 45 straighten up into a straight angle (relative to the center rod 143). The tubular container is removed over the plunger handle that is bent parallel to the gel container top. The pressing surface is releasing into a cross bar position thus forming with the handle a rotating stirring crank, rotating the stirring arms with the handle thus, mixing the powder and gel unit doses thoroughly. When the flap cover 54 is opened the rotation of the arms propels the mixed and activates gel-powder mixture out of the outlet port. In other configurations the powder container may be an integral part of the vehicle container (inside or outside) with the plunger system designed to open the communication between the two containers, mix the components and expels the mixture.

### DETAILED DESCRIPTION OF THE PREFERRED EMBODIMENT

The invention can be carried out using a variety of systems and means, one of which is described hereinafter in detail for the purpose of illustration. The different components of a system according to one embodiment of the invention, which is useful for the new comprehensive treatment made possible by the invention are:

1. Pre- and post-debridement, sets.

2. Debridement sets.

3. Early coverage sets.

4. Late grafting sets.

5. General dressing sets.

These elements will be described in greater detail hereinafter.

1. Pre- and Post-Debridement Preparation Sets

This preparatory set is designed to provide specific means for the treatment of the traumatized skin before and after debridement. The goal of this treatment is to preserve as much as possible of the living tissues in the harsh conditions of a traumatized skin with impaired local circulation at the periphery of the dead eschar and the denuded wound's bed after the debridement of the dead eschar.

The set that provides the protective micro-environment may be composed of an occlusive or open, non-occlusive dressing. An occlusive dressing such as the Multipurpose Dynamic Occlusive Dressing (M.D.O.D.), (which is the subject of a copending patent application filed by the same applicant herein, on the same day as this application and identified as Attorney Docket no. 4132/96) may provide all the changing, dynamic needs of the wound but a combination of different occlusive and non occlusive dressings may also fulfill the physical and chemical needs. In principle, the first needs are to humidify the dry, traumatized tissues (dry eschar) and to provide the necessary moisture to the surrounding tissues. A water containing hydrating dressings, gel, soaking dressing, ointments or creams may be used. The use of these dressings inside the M.D.O.D. or a traditional occlusive chamber increases the efficacy and the bioactivity of the various components and a special attention should be paid not to surpass the therapeutic phase and in some cases, even harming the sensitive tissues. In the case of non-occlusive dressings the danger is usually the desiccation of the dressing and the adjacent tissues with sometimes an adverse effect of the increased concentration of the solutes on the wound. In such cases a change or correction of the dressing is mandatory. The M.D.O.D allows a minute control of the occlusive chamber ambient and its continuing changes and electrical and ionophoretic enhancement of the process according to need. The following alternative dressings may be used as pre and post-debridement environmental dressings.

1. An occlusive dressing as previously described.

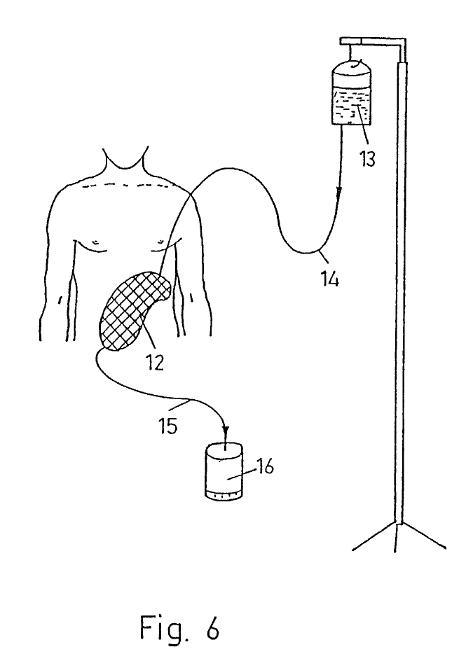

2. A continuous irrigation/soaking dressing. A thick gauze or fibrous knotted (Kerlix type) dressing with an irrigation tube that is imbedded in the dressing and allows a continues irrigation with the desired liquid (FIG. 6).

3. A traditional heavy gauze or knotted dressing that is soaked with desired liquids at the desired intervals.

2. The Debridement Set.

The debridement process is designed to produce within few hours a wound bed, clean of dead eschar and covered with the above mentioned interface layer (I.L.).

The debridement timing is important. The older the eschar is, the more susceptible it is to contain contaminating germs. The debridement process (whether surgical or chemical) introduces these germs with their toxic products into the blood stream causing bacteremia and toxemia. The longer the debridement process, the more germs and toxic materials are inoculated.

In order to prevent this bacteremia and toxemia the debridement should be performed on the freshest possible eschar and the process should be as short and as fast as possible.

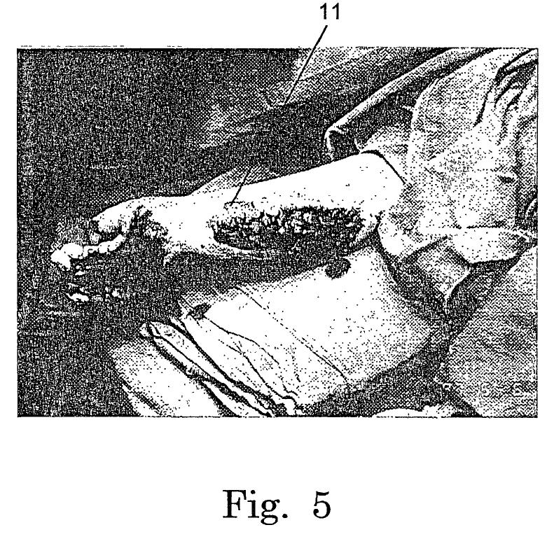

It is seen in photo no. 2 that the I.L. (numeral 4) is the first tissue layer immediately adjacent to the traumatized tissue and is characterized by a normal microscopic appearance of the various structures, especially the collagen fibers but most of the patent, functioning blood vessels are not transacted by the selective debridement process. At the level that most traumatized vessels are debrided they are still occluded by thrombi or vasoconstriction at their end. This differs from a surgically debrided tissue where the level of transaction is within a healthy tissue and the vessels are transacted and bleed freely (FIG. 5) until the natural or artificially assisted hemostasis phenomenon take place. The macroscopic representation is of a whitish layer with few (compared to a surgically debrided tissue) bleeding points (Photo nos. 2,3,4, numeral 3,4,7,8,9,10). This layer is able to support the first imbibition phase of skin graft “take” where the exudate serum from the raw surface nourishes the graft. In this phase the raw dermal side of the graft adheres to the debrided surface, protects it and serves as a matrix for the propagation of the remaining epitlielial (epidermal keratocytes) cells that survived in the skin adnexa and at the wounds perimeters. In spite of what was said before, in many cases (especially with thin, partial thickness skin grafts) the I.L. may also support the second stage of skin graft take:

The neo-vascularization phase that is the anastomosis of some of the opened blood vessels with some of the grafts vessels and the ingrown of capillary endothelial buds into the graft. Meshing the graft (inserting it in a mesh form) for expansion and drainage may also enhance the healing process. Nevertheless, the main objective of the treatment modality is to promote first the spontaneous healing of the wound by epithelialization and not to graft it permanently. The autogeneous grafting procedure is reserved only to full thickness wounds without any dermal remains that could not be epithelialized within 2-4 weeks.

General Procedures

The specific method of producing the I.L. by a chemical or enzymatic debridement will now be fully explained through a standard in vivo, bioassay test, comprising the following steps:

1. An anesthetized 10 kg. piglet is used for the bioassay. Its back hair is clipped, the hair should not be shaved or dissolved with epilating products, in order not to change the skin integrity and fine structure. Radiant, contact and scald burns are inflicted in order to produce 5×5 centimeters mixed depth burns where the center of at least 2×2 centimeters are of a full thickness burn and the rest gradually bevels to a superficial burn. Such burn imitates most of the clinical conditions. Ten burns for each etiologic agent, symmetrically placed five on each side are inflicted.

2. According to the specific debriding agent the debridement procedure may change. The debriding agent should have the following characteristics:

Does not harm the healthy or untraumatized tissues.

Does not have toxic side effects in the prescribed uses.

Fast action (very few hours; less than 12, preferably less than 4 hours).

Throughout this specification Debridase (Bromalein) (described, e.g., in U.S. Pat. No. 4,226,854) is used as an example for the debriding agent, it being understood that the invention is in no way limited to any specific debriding agent. However, as will be appreciated by the skilled person the amount and the concentration of the debriding agent in the debriding composition may change from one debriding agent to another. For this reason, the appropriate interface layer-forming effective amount of debriding agent should be determined in each case, using the standard test described herein, or a comparable test.

After removing epithelial blisters a saline soaking dressing (such as previously described) is applied on the burns for two hours. After the soaking the remaining epithelial blisters and burned epidermis are removed by rubbing it with a saline wet gauze. An adherent barrier is applied around the burns including 1 centimeter of healthy, undamaged skin as first step for the MDOD occlusive dressing. The burn is sprinkled with warm saline at 37 centigrade or covered with thin layer of hydrating gel and the dry Debridase is applied in unit dose of descending values.

The unit doses may be achieved by using a unit dose debriding matrix or by using unit dose powder sprinklers such as described later. It is possible to mix a predetermine amount of dry Debridase in its hydrating gel using a device as previously described but in this case the amount of enzyme that is in actual contact with the eschar is hard to determine. It is important to be able to determine the exact quantity of the debriding agent in order to find its efficacy. When using the Debridase of U.S. Pat. No. 4,226,854, an amount of 2 grams for 100 cm² burn was used.

The dry enzyme is sprinkled with 37 centigrade warm saline (5 cc. for each 100 cm²) covered with 25 cc. of hydrating gel. (according to U.S. Pat. No. 4,226,854) and the occluding film is applied over the adherent barrier in tight contact with the debriding agent in order to exclude air. When using the M.D.O.D., the air may be sucked out after closure of the film. Special care is taken to keep the piglet back temperature at 37 centigrade.

After 4 hours the dressings are removed, the gel and the enzyme with or without the carrier matrix are wiped away and the treated areas are vigorously scraped using dry gauze, 20 times for each burn. The burns are assessed and photographed. At this stage the overdebrided areas will show a profuse bleeding from many vessels. A bleeding from very few bleeding vessels does not mean overdebridement. The I.L. will show as whitish layer with an pink color showing from underneath and several punctuate, very slowly bleeding points. This will happen wherever dermis exist. At the center, where the burn is full thickness, the exposed fat with or without bleeding vessels shows. The underdebrided burns will appear as areas with white or gray, partially digested eschar. Between such island of undebrided eschar several areas of I.L. may exist, as represented and described in photographs 2, 3 and 4.

The debrided areas are soaked as previously described for 2-4 hours. After this post-treatment soaking the wound is reassessed for the presence of I.L., eschar, bleeding vessels, exposed fat or deeper tissues.

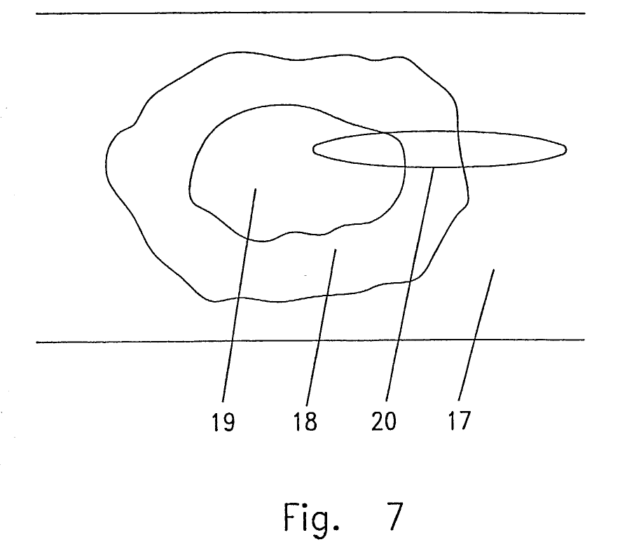

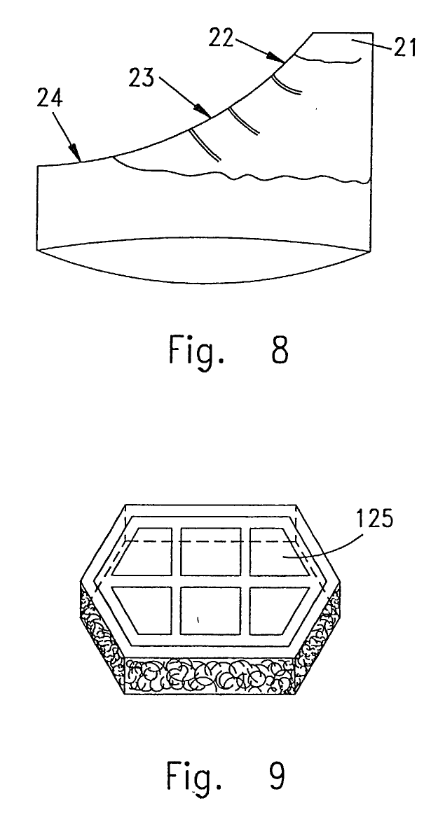

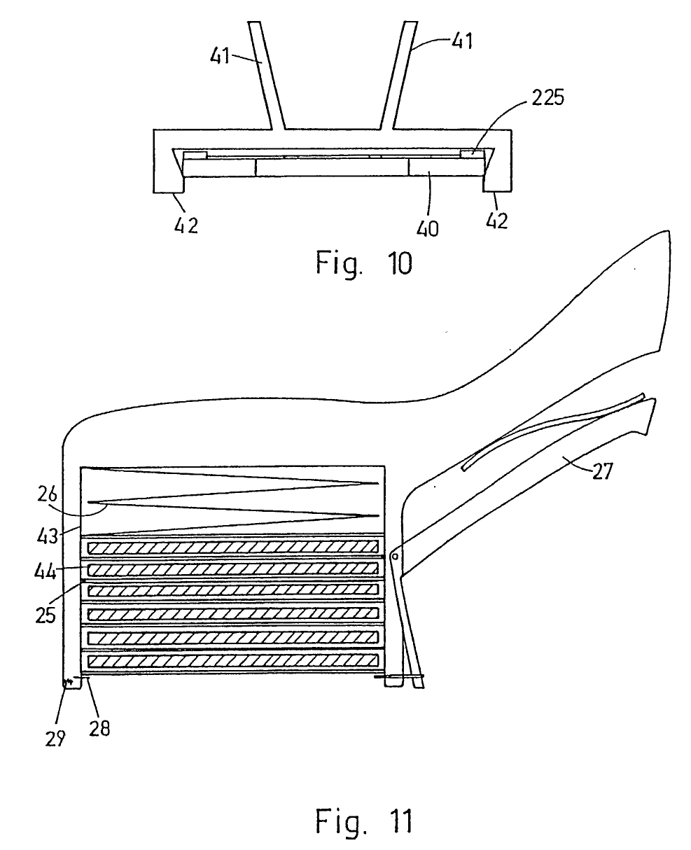

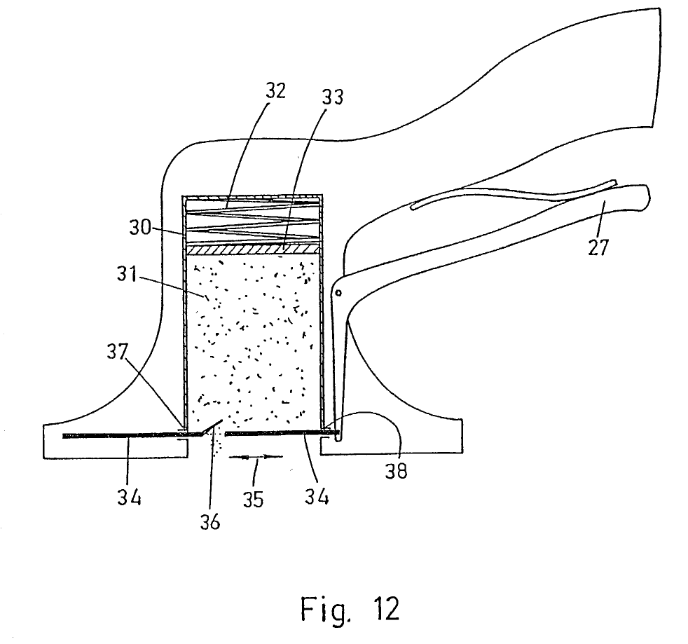

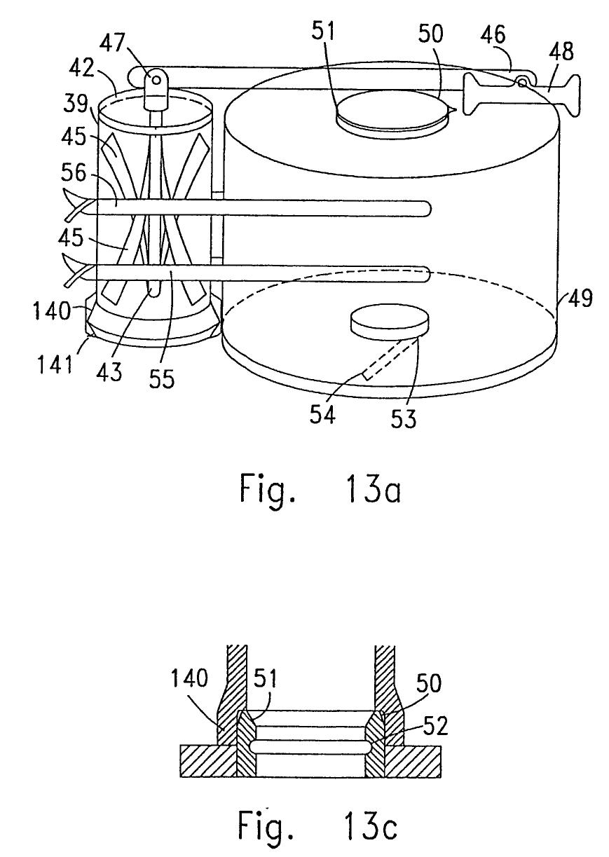

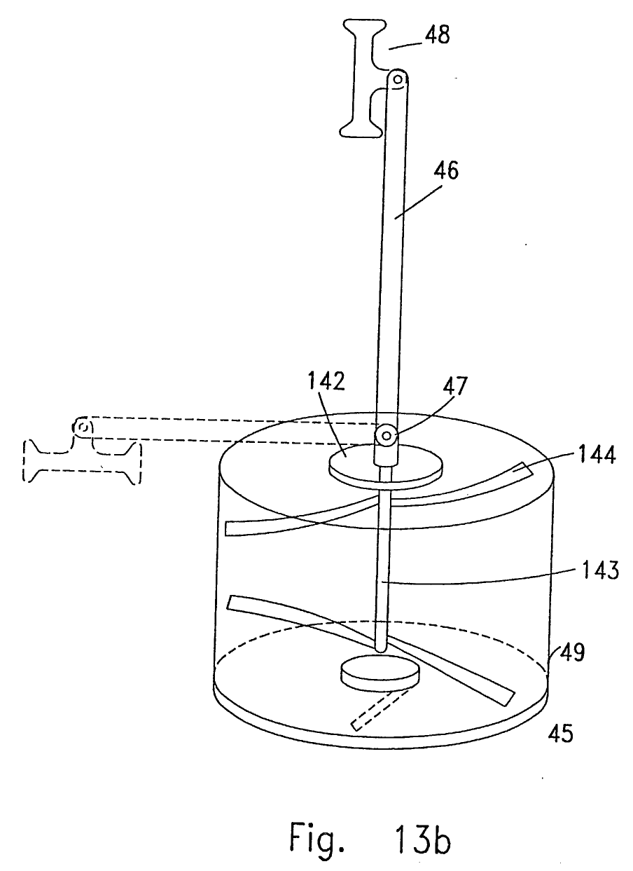

The reassessment is confirmed by a radial inscisional biopsy containing at one end the healthy intact skin and at the other end the deepest wound at the center of the test area as represented and described in FIGS. 7 and 8. The clinical application of the debriding set is essentially the same, using predetermined quantities of debriding agent in occlusive dressing after removal of the burned epithelium and blisters and soaking.

3. Early Coverage Set

The goal of the early cover is to promote a fast, spontaneous epithelialization of the debrided wound by providing the optimal physical, chemical and hormone factors needed for the remaining dermal and epidermal components that were preserved in the debridement process.

The early cover for the debrided wound should provide the following features:

1. Adherence: The dressing should adhere intimately to the wounds bed in order to provide the right survival conditions for the exposed raw tissues. The adherence preserves adequate humidity and protects against desiccation, contamination and propagation of infection. The adherent surfaces provide some of the necessary condition for the Keratocytes propagation.

2. Matrix for Keratocytes (epithelial cells) propagation: The dressing should enhance or be the matrix for the multiplication and propagation of the epithelial cells. These cells will originate within the skin remnants and/or may be imported to the wound's site from other areas of the patient (autogeneous graft) or other patients (omograft). The natural Keratocyte's matrix is the dermis thus, any graft containing dermis that will not provoke an immune response from the host may be appropriate. An artificial “dermis” made of various collagen fibers may under certain circumstances serve as such a matrix (e.g. Ortec's Composite Cultured Skin—Ortec CCS, Integra artificial dermis etc.).

3. Wound healing and epithelialization enhancement: The right hormone growth factors applied in the right amount and sequence is essential for an optimal epithelialization process. Though, many of the factors are known and some of them even synthesized, the exact combination and sequence of the entire hormone system is still unknown. One way to overcome this problem is to graft onto the healing wound an exogenous source that will produce the entire hormone system. This “hormone factory” is the epithelial cell ,(Keratocyte) itself and grafting these cells either as omograft, Keratocyte culture, cell suspension or combined biological dressing that contain Keratocytes and a collagen matrix may serve this purpose.

The coverage set includes a biological cover with the above mentioned features and any necessary devices, instruments or dressings (some of these may be part of the late grafting set). An example for such a coverage is the omograft (a partial thickness skin graft of human donor) in the form of plain sheathes or meshed. The Ortec CCS is a semi synthetic “omograft” made of an artificial collagen layer (serving as a dermis) and live, donor's Keratocytes suspension. In both cases the dressing's collagen layer provides the physical condition for protection and healing of the debrided wound and the living cells the hormones and growing factors for the Keratocytes multiplication and propagation.

Other biological covers may include the Integra artificial dermis, Seprafilm (Genzyme), and preparations combined with living cells such as Dermagraft-TC, (Advanced Tissue Science—ATS; La Jolla, Calif.), Cariel (Medical Sciences Inc. Princeton, N.J.), Apligraft (Organogenesis, Canton, Mass.), Adcon-T.N. and Adcon-L (Gliatech inc. Cleveland).

The combination of the I.L. and the coverage is the key for the optimal healing process, where by the end of 2-4 weeks large parts or even most of the burn are covered by newly formed epithelium on a collagen (dermal) foundation and the coverage remains sloughed off.

The process of Keratocytes propagation and wound healing enhancement may demand a dressing that will provide the adequate cover, support and protection to the healing wound but will not interfere with its dynamic behavior. The traditional cottonwool derivatives such as the gauze provide good cover and propagation environment but the Keratocytes and the granulation tissue tend to grow into the fibers and fibers become imbedded in to the healing and growing tissues. Dressing changes besides being a traumatic experience to patients and personnel, disrupts the healing process. It has been found, and this is a furthere aspect of the invention that a silicon impregnated dressing provides all the benefits of the gauze, without its drawbacks by not allowing the healing tissues ingrowth.

In several cases of extensive, deep burns some areas may still show raw bed or with the beginning of development of granulation tissue. These areas may need an autogeneous skin grafting (autograft) for complete closure or be left for spontaneous healing (by secondary intention) and scarring.

4. Late Grafting Set.

The late grafting set is designed to provide the means for grafting the areas that were not healed by the early debridement and enhanced healing procedure previously described. The grafting technique and the use of devices such as prep razor/disposable dermatomes, manual or powered dermatomes (for skin graft harvesting), manual or powered meshers is well rooted in the daily practice of wound treatment (U.S. Pat. No. 4,690,139 and patent application PCT/IL96/00174).

As the healing of the grafted wound depends very much on the same factors as the enhanced healing of the covered I.L., a similar approach may be used here as well. Basically the wound bed is clean without dermal or epidermal remnants that could be used as healing foci. An autograft containing autogeneous dermis and Keratocytes is imported and spread over the recipient bed. In cases where a large area should be covered, in order to save healthy donor skin the small skin grafts may be meshed and thus expanded (Patent Application PCTIIL96/00174). The healing process and the final results of meshed grafts are not as good as with plain sheet grafts. The use of a healing enhancing coverage as previously described over a meshed autograft speeds the healing time and the end results are much better than without it. The use of early covers as previously described allows the use of a very widely meshed autografts with a farther save of donor skin. Such a cover can be used as a carrier for the meshed autograft for an easier handling and application on the recipient site. A wound enhancing coverage of the omograft or Ortec CCS type will serve not only as a physical carrier and stabilizer for the meshed autograft but by performing all the roles of an early cover for a debrided wound previously mentioned, will speed the epithelialization of the mesh defects. A fast epithelialization will reduce scarring and will lead to a better function and aesthetic results.

5. General Dressing Set.

A component of the late healing process is the epithelialization and the scar modulation phenomenon. The healing enhancing covers (such as the omograft and the Ortec CCS) may serve as epithelialization dressings but they are expensive and in many cases a less costly general dressing set may be used as dressing for the epithelializing wound. This dressing should be able to serve as a matrix for the epithelialization process without being incorporated and/or interfering with it. Such a dressing may be of the film type (such as the Omiderm or Opsite type), medicated gauze (such as the Sofratule or Rafuracin gauze) or the specially prepared silicon-impregnated dressing that is part of this invention and previously described.

All the above description of preferred embodiments has been provided for the purpose of illustration, and is not intended to limit the invention. Many modifications can be made in the various materials and methods employed. For instance, different debriding agents can be used, or different dressings and Keratocyte growth promoting agents can be employed, all without exceeding the scope of the invention.

1. A skin pre-healing composition, for the pre-treatment of traumatized skin, comprising an interface layer-forming effective amount of a debriding agent.

2. A composition according to claim 1, wherein the debriding agent is present in an amount that does not substantially harm untraumatized tissue, does not induce substantial bleeding after debridement is completed, and does not substantially remove healthy collagen or other healthy tissues.

3. A composition according to claim 1, wherein the debriding agent comprises one or more enzymes.

4. A composition according to claim 3, wherein the enzyme is a proteolitic enzyme.

5. A composition according- to claim 3, wherein the debriding agent comprises a material selected from among maleic acid, collagenase, Trypsin, Fibrinolisin-desoxyribonuclease, Sutilain, Streptokinase-streptodornase, Papain, Bromelain, Debridase, Escharase and Ananain, or a mixture of two or more of said materials.

6. A composition according to claim 3, wherein the debriding agent is derived from pineapple.

7. A composition according to claim 6, wherein the debriding agent is Bromelain or a derivative or fraction thereof.

8. A composition according to claim 7, wherein the debriding agent is Debridase or Escharase.

9. An early coverage set for promoting the healing of an interface layer of a wound debrided by a composition of any one of claims 1 to 8, comprising a protective dermis-like dressing made of collagen or collagen derivatives or of human or animal dermis or dermis derivatives.

10. An early coverage set for promoting the healing of an interface layer of a wound debrided by a composition of any one of claims 1 to 8, comprising a protective dressing provided with Keratocyte growth promoting agent(s).

11. A set according to claim 10, wherein the Keratocyte growth promoting agent comprises an artificial dermis.

12. A set according to claim 10, wherein the dressing is made of a non-autogeneous graft (omo- or zynograft).

13. A set according to claim 10, wherein the Keratocyte growth promoting agent comprises one or more growth hormones.

14. A method for treating a patient suffering from trauma of the skin, comprising the steps of:
(a) pre-treating the wound by humidification;
(b) treating the wound with a debriding agent in an amount and for a period of time that do not promote substantial bleeding, and which generate an interface layer, as defined herein;
(c) covering the debrided wound with a matrix which protects the interface layer and promotes keratocytes propagation for a period of time sufficient to permit spontaneous healing of the interface layer; and
(d) grafting areas of deeper wound which were not healed through keratocytes propagation as described in (c) above.

15. A method according to claim 14, wherein Keratocyte propagation is allowed to proceed for about 2 to 4 weeks.

16. A method according to claim 14, wherein the debridement procedure is carried out for a period of time that does not exceed 12 hours

17. A method according to claim 14, wherein Keratocyte propagation is allowed to proceed for about 2 to 4 weeks.

18. An impregnated, unite, silicon gauze whereby the gauze does not adhere to the wound and promotes Keratocyte propagation along its fibers on the debrided wound or interface layer.

19. A unit dose debriding matrix carrier, comprising a housing containing lyophilized or otherwise dried debriding agent, said housing being made of porous material so as to allow passage of the debriding agent therethrough when a liquid is applied thereto, said carrier further having a polygonal shape such that a plurality of identical carriers can be placed around it, to fill a wound area.

20. A placing device for a unit dose debriding matrix carrier, comprising elastic holding means to hold the matrix in place within the device and to release the carrier when a light pressure is applied on the device.

21. A device according to claim 21, which comprises a container for a plurality of debriding matrix carriers, and means for releasing only one carrier each time a pressure is applied on the device.

22. A placing device for a powder debriding material comprising an ergonomic holding handle, a powder debriding agent container and means for activating a unit dose separation and deposition mechanism such as peeling off an accurate quantity of the powder and releasing it on a given surface area asepticly.

23. A unit dose powder/vehicle-carrier-gel mixing and placing device comprising unit dose powder and vehicle containers and means for joining said containers and mixing the countenance asepticly and extracting the mixture onto the wound.

24. a method for treating trauma of the skin, essentially as described and illustrated.
